(19) United States
(12) Patent Application Publication
(10) Pub. No.: US 2012/0171700 A1
(43) Pub. Date: Jul. 5, 2012

(54) CELL IDENTIFYING APPARATUS AND CELL IDENTIFYING METHOD

(75) Inventors: Toru Takahashi, Tokyo (JP); Ken Tsukii, Tokyo (JP); Jie Xu, Tokyo (JP)

(73) Assignee: FURUKAWA ELECTRIC CO., LTD., Tokyo (JP)

(21) Appl. No.: 13/342,245

(22) Filed: Jan. 3, 2012

Related U.S. Application Data
(63) Continuation of application No. PCT/JP2010/055812, filed on Mar. 31, 2010.

(51) Int. Cl. G01N 21/64; C12M 1/34

(52) U.S. Cl. 435/7.23; 435/288.7; 435/287.2; 435/34

(57) ABSTRACT

A cell identifying apparatus includes a capillary through which a sample flow passes, an irradiating unit that irradiates irradiation light, a light receiving unit that receives transmitted light and outputs a transmitted light signal based on the transmitted light, a measuring unit that receives the transmitted light signal output from the light receiving unit and measures the change of the transmitted light signal caused by cells, an identifying unit that identifies whether the cells are peripheral blood cells on the basis of a value of an extreme point in the change of the transmitted light signal, the change being caused by the cells and measured by the measuring unit, a sorting unit that sorts the cells in a predetermined vessel, on the basis of the identification result of the identifying unit, and a supplying unit that supplies the sample flow to the capillary.

### TECHNICAL FIELD

The present invention relates to a cell identifying apparatus and a cell identifying method. In particular, the present invention relates to a cell identifying apparatus and a cell identifying method that identify circulating cancer cells dispersed in a liquid flowing through a flow passage, on the basis of the change in a transmitted light signal.

### BACKGROUND ART

There is suggested a technology of allowing a liquid in which samples (minute samples to be tested) are dispersed to flow through a capillary, irradiating light emitted from a light source onto the liquid flow, and obtaining optical information (fluorescent information) on the samples in the liquid flow (for example, refer to Patent Document 1).

In recent years, as a method for early detection of a cancer, a method of detecting cancer cells that have spread to peripheral blood, that is, circulating cancer cells has drawn attention. For example, there is suggested a method of identifying only cancer cells of a breast cancer in the peripheral blood by magnetically separating cancer cells of a breast cancer labeled with minute iron beads using an antibody/antigen reaction on the cell surface and further labeling white blood corpuscles, which are merely incorporated, with activated protein C (APC) to remove the white blood corpuscles,

### PRIOR ART DOCUMENT

### Patent Document

Patent Document 1: Japanese Patent No. 2,973,387

### DISCLOSURE OF THE INVENTION

### Problems to be Solved by the Invention

However, in the above-described method of detecting the circulating cancer cells using the antibody/antigen reaction on the cell surface, different antibodies to cope with different cancers, such as breast cancer and stomach cancer, need to be developed. Currently, there are problems such that only antibody for the breast cancer has been developed but antibodies for other cancers have not yet developed, so other antibodies should be developed depending on types of cancers.

Accordingly, the present invention has been made to solve the above-described problems, and an object of the invention to provide a cell identifying apparatus and a cell identifying method for identifying circulating cancer cells that do not involve an antibody/antigen reaction of a cell surface.

### Means for Solving the Problems

The inventors examined the above problems thoroughly, has discovered that, when a liquid in which cells in peripheral blood are dispersed is irradiated with light, circulating cancer cells can be identified on the basis of a value of an extreme point of the change of transmitted light signal, the change being caused by cells, and has completed the present invention.

Here, the term “transmitted light” refers to light received by a light receiving unit, such as light transmitted through the liquid in which the cells are dispersed, light transmitted through the cells, and light reflected, scattered and diffracted by the cells and the term “transmitted light signal” refers to a signal that is obtained by converting the transmitted light into an electric signal. An arbitrary area configured to receive the transmitted light always receives light. However, light reception power (transmitted light signal) changes when the cells are measured. A peak, a width, and an area of this change are called transmitted light information.

That is, a cell identifying apparatus according to a first aspect of the present invention is a cell identifying apparatus that identifies different cells or cell species in peripheral blood. The cell identifying apparatus includes an irradiating unit that irradiates single-mode light having constant power as irradiation light onto a liquid in which the cells in the peripheral blood are dispersed, a light receiving unit that is provided at the position facing the irradiating unit and receives transmitted light through the liquid based on the irradiation light that is emitted from the irradiating unit and irradiated onto the liquid in a state in which the relative positions of the cells with respect to the irradiation light change at a constant speed, and outputs a transmitted light signal based on the transmitted light, a measuring unit that receives the transmitted light signal output from the light receiving unit and measures the change of the transmitted light signal caused by the cells, and an identifying unit that identifies the cells in the peripheral blood, on the basis of a value of an extreme point in the change of the transmitted light signal caused by the cells, which is measured by the measuring unit.

In this case, the peripheral blood cells are white blood corpuscles, red blood corpuscles, and blood platelets that are the cells existing in the peripheral blood, and the cells in the peripheral blood are the white blood corpuscles, the red blood corpuscles, and the blood platelets corresponding to the peripheral blood cells and the cells other than the peripheral blood cells (cells other than the white blood corpuscles, the red blood corpuscles, and the blood platelets). The white blood corpuscles are divided into lymphocytes, monocytes, and granulocytes. The term “different cell species” means that types of the cells are different from each other, and the term “different cells” means that cells are the same in the type but are different cell states such as cell cycles and thus they are identified as different ones.

According to a cell identifying apparatus in accordance with a second aspect of the present invention, in the cell identifying apparatus according to the first aspect of the present invention, when a value of the transmitted light signal obtained based on the liquid having no cells is set as a reference value, the identifying unit compares a value of a maximum point in the change of the transmitted light signal caused by the cells and the reference value, and identifies the cells as cells other than peripheral blood cells if the value of the maximum point is greater than or equal to the reference value.

In the change of the transmitted light signal, the cells are identified as cells other than the peripheral blood cells, when the value of the maximum point is greater than or equal to the reference value. However, since the transmitted light signal has the reference value at the beginning and ending of the measurement, this is not included. In this case, when the value of the maximum point is greater than the reference value, the cells may be identified as the cells other than the peripheral blood cells, and the reference value at the beginning and ending of the measurement may not be identified.

According to a cell identifying apparatus in accordance with a third aspect of the present invention, in the cell identifying apparatus according to the first or second aspect of the present invention, the cells that are identified as the cells other than the peripheral blood cells by the identifying unit are circulating cancer cells.

The circulating cancer cells are cancer cells that spread to the peripheral blood.

According to a cell identifying apparatus in accordance with a fourth aspect of the present invention, in the cell identifying apparatus of any one of the first to third aspects of the present invention, the cells are cells on which fluorescent processing including an antibody/antigen reaction of a cell surface and expression of fluorescent proteins in the cells is performed.

According to a cell identifying apparatus in accordance with a fifth aspect of the present invention, in the cell identifying apparatus of any one of the first to fourth aspects of the present invention, the measuring unit measures transmitted light information based on the change of the transmitted light signal, and the identifying unit identifies the cells in the peripheral blood on the basis of the transmitted light information measured by the measuring unit.

According to a cell identifying apparatus in accordance with a sixth aspect of the present invention, the cell identifying apparatus of any one of the first to fifth aspects of the present invention further includes a second light receiving unit that is provided at the position other than the position facing the irradiating unit and receives scattered light and/or fluorescent light of the cells based on the irradiation light irradiated by the irradiating unit and outputs a scattered/fluorescent signal based on the scattered light and/or fluorescent light. The measuring unit receives the scattered/fluorescent signal output from the second light receiving unit and measures the change of the scattered/fluorescent signal caused by the cells, and the identifying unit identifies the cells in the peripheral blood, on the basis of the change of the scattered/fluorescent signal measured by the measuring unit.

According to a cell identifying apparatus in accordance with a seventh aspect of the present invention, in the cell identifying apparatus of any one of the first to sixth aspects of the present invention, the measuring unit measures scattered/fluorescent information based on the change of the scattered/fluorescent signal, and the identifying unit identifies the cells in the peripheral blood on the basis of the scattered/fluorescent information measured by the measuring unit.

According to a cell identifying apparatus in accordance with an eighth aspect of the present invention, in the cell identifying apparatus of any one of the first to seventh aspects of the present invention, the irradiation light is non-condensed single-mode light.

According to a cell identifying apparatus according to a ninth aspect of the present invention, the cell identifying apparatus of any one of the first to eighth aspects of the present invention further includes a sorting unit that sorts the cells in the peripheral blood identified by the identifying unit in a predetermined vessel.

A cell identifying method according to a first aspect of the present invention is a cell identifying method that identifies cells in peripheral blood using an irradiating unit that irradiates single-mode light having a constant power as irradiation light onto a liquid in which the cells in the peripheral blood are dispersed, and a light receiving unit that is provided at the position facing the irradiating unit, receives transmitted light, outputs a transmitted light signal based on the transmitted light to a measuring unit. The cell identifying method includes a step of irradiating, with the irradiation light from the irradiating unit, the liquid in which the relative positions of the cells with respect to the irradiation light change at a constant speed, (b) a step of receiving transmitted light through the liquid based on the irradiation light by the light receiving unit and outputting a transmitted light signal based on the transmitted light to the measuring unit, (c) a step of receiving the transmitted light signal output in the step (b) by the measuring unit and measuring the change of the transmitted light signal caused by the cells with the measuring unit, and (d) a step of identifying the cells in the peripheral blood, on the basis of a value of an extreme point in the change of the transmitted light signal caused by the cells, which is measured in the step (c).

According to a cell identifying method in accordance with a second aspect of the present invention, in the cell identifying method according to the first aspect of the present invention, when a value of the transmitted light signal obtained based on the liquid having no cells is set as a reference value, in the step (d), a value of a maximum point in the change of the transmitted light signal caused by the cells and the reference value are compared, and the cells are identified as cells other than peripheral blood cells when the value of the maximum point is greater than or equal to the reference value.

According to a cell identifying method in accordance with a third aspect of the present invention, in the cell identifying method of the first or second aspect of the present invention, the cells that are identified as the cells other than the peripheral blood cells in the step (d) are circulating cancer cells.

According to a cell identifying method in accordance with a fourth aspect of the present invention, in the cell identifying method of any one of the first to third aspects of the present invention, the cells are cells on which fluorescent processing including an antibody/antigen reaction of a cell surface and expression of fluorescent proteins in the cells is performed.

According to a cell identifying method in accordance with a fifth aspect of the present invention, in the cell identifying method of any one of the first to fourth aspects of the present invention, in the step (c), transmitted light information is measured based on the change of the transmitted light signal, and in the step (d), the cells in the peripheral blood are identified on the basis of the transmitted light information measured in the step (c).

According to a cell identifying method in accordance with a sixth aspect of the present invention, in the cell identifying method of any one of the first to fifth aspects of the present invention, a second light receiving unit that is provided at the position other than the position facing the irradiating unit and receives scattered light and/or fluorescent light of the cells based on the irradiation light irradiated by the irradiating unit and outputs a scattered/fluorescent signal based on the scattered light and/or fluorescent light to the measuring unit is further used, and in the step (b), the scattered light and/or fluorescent light of the cells based on the irradiated light are/is received by the second light receiving unit and a scattered/fluorescent signal based on the scattered light and/or fluorescent light is output to the measuring unit, and in the step (c), the scattered/fluorescent signal output from the step (b) is received by the measuring unit and the change of the scattered/fluorescent signal caused by the cells is measured with the measuring unit, and in the step (d), the cells in the peripheral blood are identified on the basis of the change of the scattered/fluorescent signal measured in the step (c).

According to a cell identifying method in accordance with a seventh aspect of the present invention, in the cell identifying method of any one of the first to sixth aspects of the present invention, in the step (c), scattered/fluorescent information is measured based on the change of the scattered/fluorescent signal, and in the step (d), the cells in the peripheral blood are identified on the basis of the scattered/fluorescent information measured in the step (c).

According to a cell identifying method in accordance with an eighth aspect of the present invention, in the cell identifying method of any one of the first to seventh aspects of the present invention, the irradiation light is non-condensed single-mode light.

According to a cell identifying method in accordance with a ninth aspect of the present invention, the cell identifying method of any one of the first to eighth aspects of the present invention further includes (e) a step of sorting the cells in the peripheral blood identified in the step (d) in a predetermined vessel.

### Effects of the Invention

According to the cell identifying apparatus and the cell identifying method of the present invention, the cancer cells that spread to the peripheral blood, that is, the circulating cancer cells can be identified without using the antibody/antigen reaction of the cell surface.

### BRIEF DESCRIPTION OF THE DRAWINGS

FIG. 1 is a schematic side view of a cell identifying apparatus 10 according to an embodiment of the present invention.

FIG. 2 is a cross-sectional view taken along the line K-K of FIG. 1.

FIG. 3 is a diagram illustrating a characteristic of a waveform of the change of a transmitted light signal SG1 with respect to cells in peripheral blood, which is measured by a measuring unit 40 of the cell identifying apparatus 10 according to the embodiment of the present invention.

FIG. 4 is a diagram illustrating an example of the result of the change of the transmitted light signal SG1 with respect to circulating cancer cells, which is measured by the measuring unit 40 of the cell identifying apparatus 10 according to the embodiment of the present invention.

FIG. 5 is a flowchart illustrating a cell identification processing sequence using the cell identifying apparatus 10 according to the embodiment of the present invention.

FIG. 6 is a schematic side view of another cell identifying apparatus 10a according to an embodiment of the present invention.

FIG. 7 is a cross-sectional view taken along the line K-K of FIG. 6.

FIG. 8 is a flowchart illustrating a cell identification processing sequence using another cell identifying apparatus 10a according to the embodiment of the present invention.

### BEST MODE(S) FOR CARRYING OUT THE INVENTION

Hereinafter, an embodiment of the present invention will be described in detail on the basis of the drawings.

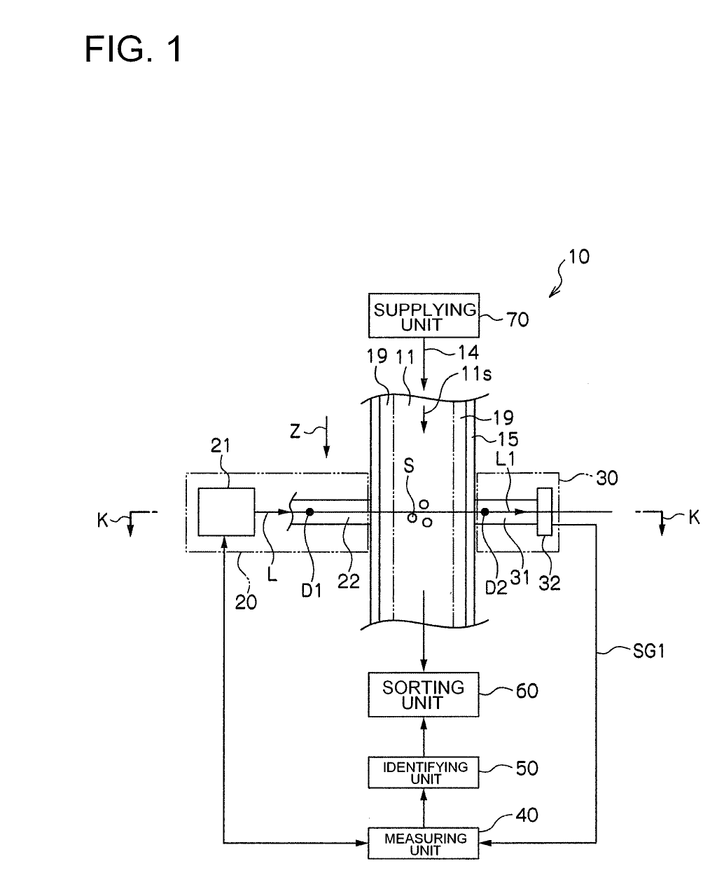

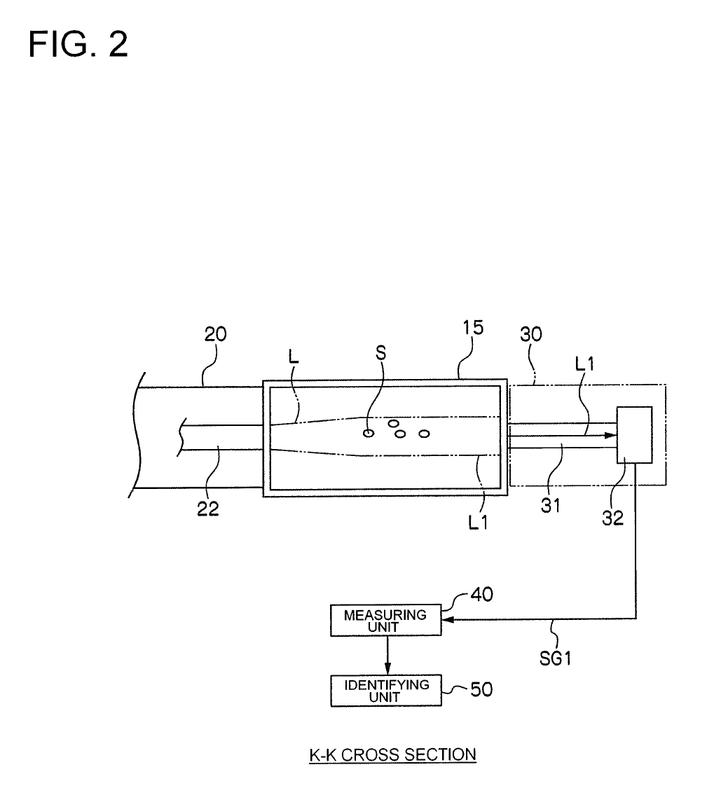

FIG. 1 is a schematic side view of a cell identifying apparatus 10 according to an embodiment of the present invention. FIG. 2 is a cross-sectional view taken along the line K--K of FIG. 1.

As shown in FIGS. 1 and 2, the cell identifying apparatus 10 according to the embodiment of the present invention includes a capillary 15 that flows a sample flow 11S, an irradiating unit 20 that irradiates single-mode light having constant power as irradiation light L, a light receiving unit 30 that receives transmitted light L1 and outputs a transmitted light signal SG1 based on the transmitted light, a measuring unit 40 that receives the transmitted light signal SG1 output from the light receiving unit 30 and measures the change of the transmitted light signal SG1 caused by cells S, an identifying unit 50 that identifies whether the cells S are peripheral blood cells, on the basis of the change of the transmitted light signal SG1 caused by the cells S, a sorting unit 60 that sorts the cells S in a predetermined vessel, on the basis of the identification result of the identifying unit 50, and a supplying unit 70 that supplies the sample flow 11S to the capillary 15.

The supplying unit 70 executes processing (hereinafter, preprocessing) for removing red blood corpuscles and blood platelets, from a liquid 11 where the cells extracted from the peripheral blood are dispersed. The cells in the peripheral blood other than the red blood corpuscles and the blood platelets that remain after the preprocessing are called the cells S. The supplying unit 70 flows the liquid 11 where the cells S are dispersed as the sample flow 11S and a sheath flow 19 in a Z direction (direction from the upper side to the lower side in FIG. 1), and supplies them to the capillary 15 through a tube 14. The supplying unit 70 adjusts pressures of the sample flow 11S and the sheath flow 19 and adjusts a flow rate and a flow volume of the cells S that are dispersed in the liquid 11 in the capillary 15.

The cells S that are dispersed in the liquid 11 are the cells that are only extracted from the peripheral blood. However, the cells S may be cells extracted from the peripheral blood, on which fluorescent processing including nuclear staining, an antibody/antigen reaction of a cell surface, and expression of fluorescent proteins in the cells is performed.

As a sequence of the preprocessing, for example, a lysing agent dedicated for human (FACS Lysing Solution) that includes both a lysing agent and a fixing agent (formaldehyde) is diluted 10 times with MilliQ (pure water) and is introduced into a test tube of 15 mL by 6.5 to 7 mL. Next, a whole blood sample (which is a suspension of red blood corpuscles and white blood corpuscles of a normal person and has hemolysis, scattered light, antigen expression, and an antibody staining property of whole blood of a normal person) of 1 mL is added to the test tube, is moderately mixed by a pipette, and is stored at a room temperature for 15 to 30 minutes. Then, the resultant is centrifugally separated at 1000 rpm for 5 minutes, a supernatant solution is sucked, phosphate buffered saline (PBS) of 5 mL is added, and resuspension is performed. The sample where the centrifugal separation and the resuspension are repeated 3 times is stored at a room temperature for 2 hours and is stored at the temperature of 4° C. for 24 hours. Finally, the sample is made to pass through a filter of a mesh of 40 μm and the preprocessing ends.

The irradiating unit 20 irradiates single-mode light having constant power as the irradiation light L onto the liquid 11 in the capillary 15. For example, as shown in FIGS. 1 and 2, the irradiating unit 20 includes a laser light source 21 and an optical fiber 22 for irradiation. As an example of the laser light source 21, a laser diode is known. The supplying unit 70 adjusts the pressures of the sample flow 11S and the sheath flow 19, such that the relative positions of the cells S with respect to the irradiation light L from the irradiating unit 20 change at a constant speed, when the irradiation light L is irradiated onto the liquid 11 in the capillary 15. In this case, the irradiation light L is preferably non-focused single-mode light. The single-mode light has a strength pattern of a Gaussian distribution.

The light receiving unit 30 is provided at the position that faces the irradiating unit 20 through the capillary 15 and receives the transmitted light L1 through the liquid 11 based on the irradiation light L and outputs a transmitted light signal SG1 based on the transmitted light to the measuring unit 40. For example, as shown in FIGS. 1 and 2, the light receiving unit 30 includes a light receiving optical fiber 31 and a light receiving element 32. As examples of the light receiving element 32, a photomultiplier tube and a photo detector are known. In this case, a first optical axis D1 of the irradiating unit 20 and a second optical axis D2 of the light receiving unit 30 are preferably aligned with each other.

The supplying unit 70 adjusts the pressures of the sample flow 11S and the sheath flow 19, such that the relative positions of the cells S with respect to the irradiation light L change at a constant speed, when the irradiation light L is irradiated onto the liquid 11 in the capillary 15. Thereby, the light receiving unit 30 receives the transmitted light L1 through the liquid 11 in a state where the relative positions of the cells S with respect to the irradiation light L change at a constant speed, and outputs the transmitted light signal SG1 based on the transmitted light to the measuring unit 40.

The measuring unit 40 receives the transmitted light signal SG1 that is output from the light receiving unit 30, measures the change of the transmitted light signal SG1 caused by the cells S, and outputs the measurement result to the identifying unit 50. In this case, the change of the transmitted light signal SG1 caused by the cells S is attributable to a light diffracting phenomenon, a light interfering phenomenon, a light absorbing phenomenon, and a light scattering phenomenon that are observed when the irradiation light L is transmitted through the cells S, due to the difference of the type, shape, magnitude, and number of materials (for example, cell nucleuses) constituting the cells S.

The identifying unit 50 identifies the cells S, on the basis of a value of an extreme point in the change of the transmitted light signal SG1 caused by the cells S, the value being input from the measuring unit 40. That is, the identifying unit 50 identifies whether the cells S are the peripheral blood cells (that is, white blood corpuscles) or the cells other than the peripheral blood cells. In this case, the cells S are identified by using only the value of the extreme point in the change of the transmitted light signal SG1. However, the value of the extreme point and the number of extreme points may be combined and the cells S may be identified by the value and the number. The detailed configuration of the identification processing will be described below.

The sorting unit 60 sorts the cells S corresponding to the peripheral blood cells in a predetermined vessel (not shown in the drawings) when the peripheral blood cells are sorted on the basis of the identification result identified by the identifying unit 50, and sorts the cells S corresponding to the cells other than the peripheral blood cells in the predetermined vessel when the cells other than the peripheral blood cells are sorted.

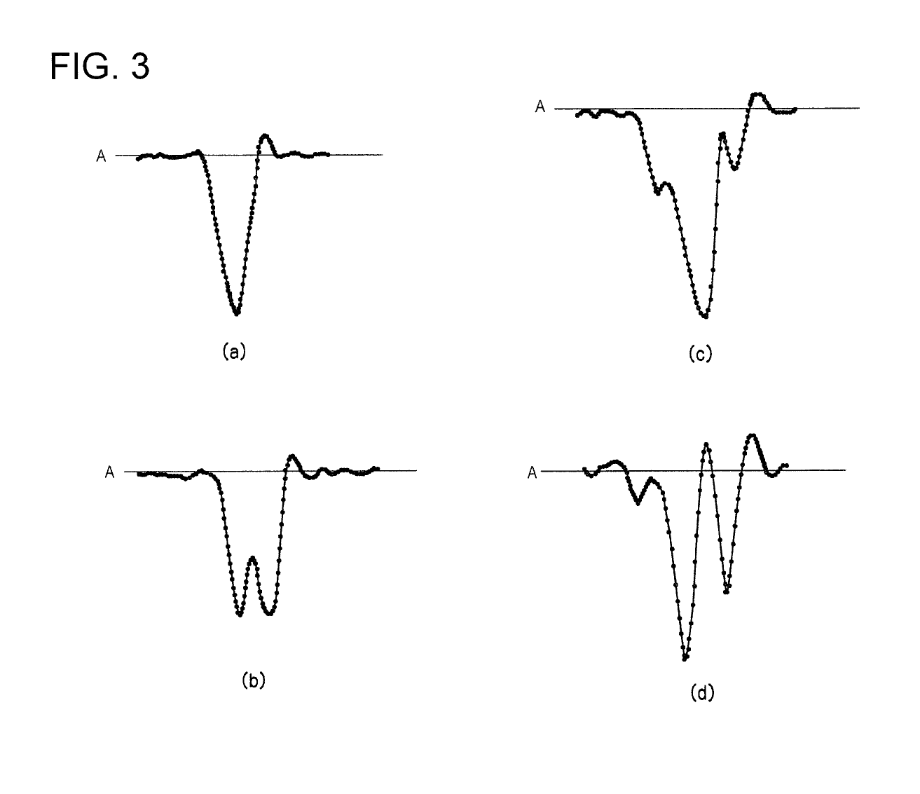

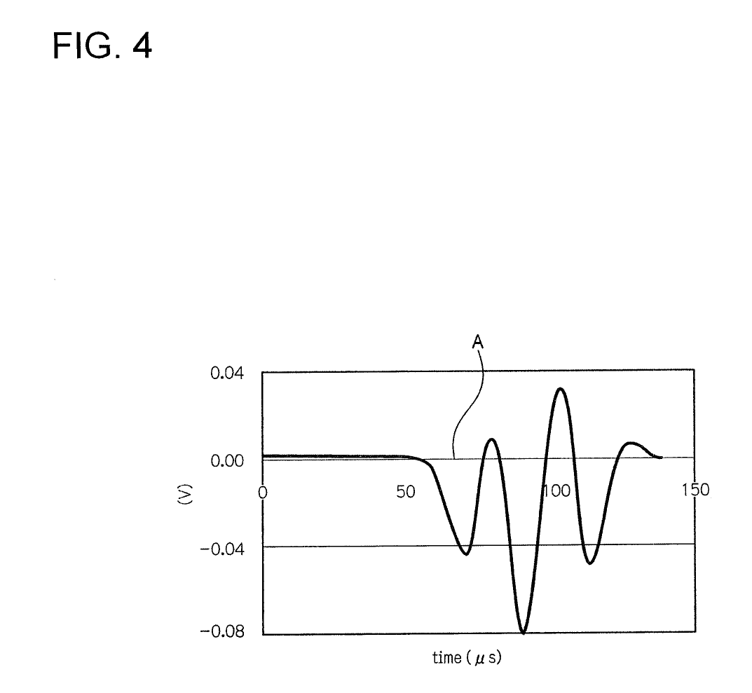

Next, the detailed configuration of the identification processing of the cells S that is executed by the identifying unit 50 of the cell identifying apparatus 10 according to the embodiment of the present invention will be described with reference to FIGS. 3 and 4 and Table 1.

FIG. 3 is a diagram illustrating a characteristic of a waveform of the change of the transmitted light signal SG1 with respect to the cells in the peripheral blood, which is measured by the measuring unit 40 of the cell identifying apparatus 10 according to the embodiment of the present invention.

As shown in FIG. 3, the waveform of the change of the transmitted light signal SG1 with respect to the cells in the peripheral blood can be sorted into four waveform patterns, which include a first waveform pattern that has one minimum point as shown in FIG. 3(a), a second waveform pattern that has two minimum points as shown in FIG. 3(b) and has a value of a maximum point equal to or smaller than a reference value A, a third waveform that has three or more minimum values as shown in FIG. 3(c) and a value of a maximum point equal to or smaller than the reference value A, and a fourth waveform pattern where a value of a maximum point is greater than the reference value A as shown in FIG. 3(d). In this case, a value of the transmitted light signal SG1 obtained based on the liquid 11 having no cells S is set as the reference value A. The change caused by overshoot that is generated in a peripheral portion after the measurement of the transmitted light signal SG1 caused by the cells S is excluded from the maximum point of the change of the transmitted light signal SG1 caused by the cells S.

When only the values of the extreme points are considered, only the fourth waveform pattern has a value of the extreme point that is greater than the reference value A, and the first, second, and third waveform patterns have values of the extreme points that are equal to or smaller than the reference value A.

Next, the measurement result of a waveform of the change of the transmitted light signal SG1 with respect to the white blood corpuscles in the peripheral blood that is measured by the measuring unit 40 of the cell identifying apparatus 10 according to the embodiment of the present invention is shown in Table 1. Table 1 shows measurement ratios (%) of the four waveform patterns with respect to white blood corpuscles in which lymphocytes, monocytes, and granulocytes are mixed, and white blood corpuscles in which only lymphocytes are mixed.

TABLE 1

| | White blood | |
|---|---|---|
| | corpuscles where | |
| | lymphocytes, | White blood |
| | monocytes, and | corpuscles where |
| | granulocytes are | only lymphocytes |
| Waveform pattern | mixed | are mixed |
| First waveform | 51.4% | 66.2% |
| pattern (refer to | | |
| FIG. 3(a)) | | |
| Second waveform | 35.3% | 32.1% |
| pattern (refer to | | |
| FIG. 3(b)) | | |
| Third waveform | 13.2% | 1.7% |
| pattern (refer to | | |
| FIG. 3(c)) | | |
| Fourth waveform | 0.0% | 0.0% |
| pattern (refer to | | |
| FIG. 3(d)) | | |

As shown in Table 1, in both the case of the waveform of the change of the transmitted light signal SG1 with respect to the white blood corpuscles where the lymphocytes, the monocytes, and the granulocytes are mixed and the case of the waveform of the change of the transmitted light signal SG1 with respect to the white blood corpuscles where only the lymphocytes are mixed, the percentage of the first waveform pattern is the largest, the percentages decrease in the order of the second waveform pattern and the third waveform pattern, and the percentage of the fourth waveform pattern cannot be measured. That is, in the waveform of the change of the transmitted light signal SG1 with respect to the white blood corpuscles, all of the values of the extreme points are equal to or smaller than the reference value A.

If a waveform of the change of the transmitted light signal SG1 with respect to cancer cells is measured by the measuring unit 40 of the cell identifying apparatus 10 according to the embodiment of the present invention, the percentage of the fourth waveform pattern is measured as 52.4% in the case of an HeLa cell (cervical cancer) and the percentage of the fourth waveform pattern is measured as 36.7% in the case of a pancreatic cancer cell. That is, in the waveform of the change of the transmitted light signal SG1 with respect to the cancer cells, the extreme point that has a value greater than the reference value A exists. When the cells in the peripheral blood are measured, when the fourth waveform pattern is detected, it is assumed that the circulating cancer cells exist in the peripheral blood in a number of two to three times the detected amount.

FIG. 4 is a diagram illustrating an example of the result of the change of the transmitted light signal SG1 with respect to the circulating cancer cells, which is measured by the measuring unit 40 of the cell identifying apparatus 10 according to the embodiment of the present invention.

From the above result, it can be seen that the cells S are the white blood corpuscle, that is, the peripheral blood cells, when the waveform of the change of the transmitted light signal SG1 with respect to the cells S in the peripheral blood is measured by the measuring unit 40 of the cell identifying apparatus 10 according to the embodiment of the present invention and the waveform pattern is any one of the first waveform pattern, the second waveform pattern, and the third waveform pattern. Further, it can be seen that the cells S are the cells other than the peripheral blood cells, that is, the cells S are the circulating cancer cells, when the waveform pattern is the fourth waveform pattern.

Therefore, the identifying unit 50 of the cell identifying apparatus 10 according to the embodiment of the present invention identifies whether the cells S are the peripheral blood cells or the cells other than the peripheral blood cells, on the basis of a value of the extreme point in the change of the transmitted light signal SG1 characterizing the four waveform patterns described above. Specifically, as shown in FIGS. 3 and 4, the identifying unit uses the value of the transmitted light signal SG1 obtained based on the liquid 11 having no cells S as the reference value A, compares the value of the maximum point in the change of the transmitted light signal SG1 caused by the cells S and the reference value A, and identifies the cells S as the peripheral blood cells (white blood corpuscles) when the value of the maximum point is equal to or smaller than the reference value A and identifies the cells S as the cells (circulating cancer cells) other than the peripheral blood cells when the value of the maximum point is greater than the reference value A.

The measuring unit 40 measures only the change of the transmitted light signal SG1 caused by the cells S. However, the measuring unit 40 may further measure transmitted light information corresponding to the peak, the width, and the area of the change of the transmitted light signal SG1, from the change of the transmitted light signal SG1. At this time, the identifying unit 50 may add and use the transmitted light information measured by the measuring unit 40 as a parameter of the identifying condition and identify the cells S.

As such, the cell identifying apparatus 10 according to the embodiment of the present invention can identify whether the cells S in the peripheral blood are the peripheral blood cells or the cells other than the peripheral blood cells without using the antibody/antigen reaction of the cell surface. That is, the cell identifying apparatus 10 can identify whether the cells S are the peripheral blood cells or the circulating cancer cells.

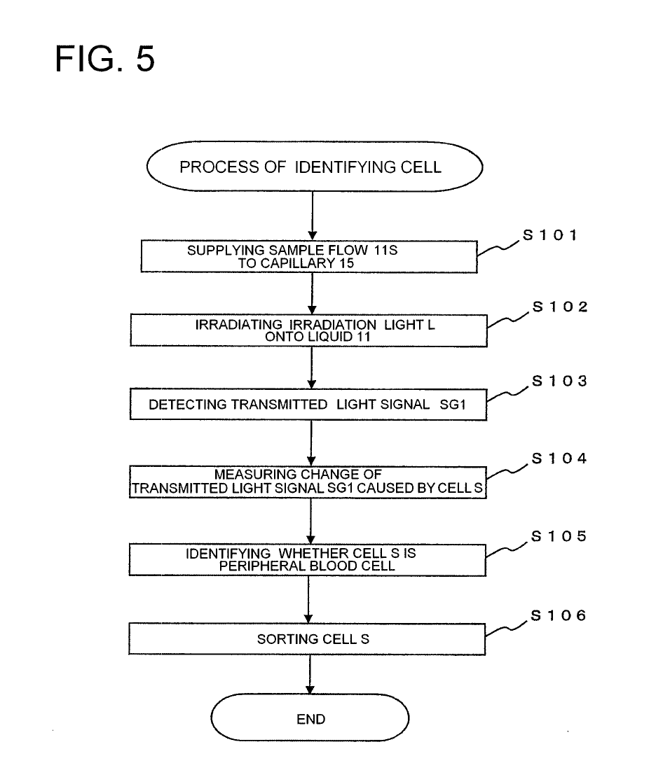

Next, a cell identification processing sequence using the cell identifying apparatus 10 according to the embodiment of the present invention will be briefly described with reference to FIG. 5.

FIG. 5 is a flowchart illustrating the cell identification processing sequence using the cell identifying apparatus 10 according to the embodiment of the present invention.

As shown in FIG. 5, first, the liquid 11 as a sample flow is supplied to from the supplying unit 70 to the capillary 15 (step 1: S101) in which the liquid is obtained by adjustment such that the relative positions of the cells S in the liquid with respect to the irradiation light L from the irradiating unit 20 change at a constant speed. The cells S are cells that are extracted from the peripheral blood where the preprocessing is performed.

Next, the irradiation light L from the irradiating unit 20 is irradiated onto the liquid 11 in the capillary 15 (step 2: S102). Next, the transmitted light L1 through the liquid 11 based on the irradiation light L is received by the light receiving unit 30 and the transmitted light signal SG1 based on the transmitted light is output to the measuring unit 40 (step 3: S103). Next, the transmitted light signal SG1 output from the light receiving unit 30 is input to the measuring unit 40 and the change of the transmitted light signal SG1 caused by the cells S is measured by the measuring unit 40 (step 4: S104).

Next, the cells S are identified on the basis of the value of the extreme point in the change of the transmitted light signal SG1 caused by the cells S, which is measured by the measuring unit 40 (step 5: S105). That is, whether the cells S are the peripheral blood cells (that is, white blood corpuscles) or the cells other than the peripheral blood cells is identified by the identifying unit 50. Specifically, when the value of the transmitted light signal SG1 obtained based on the liquid 11 having no cells S is set as the reference value A, the value of the maximum point in the change of the transmitted light signal SG1 caused by the cells S and the reference value A are compared, and the cells S are identified as the peripheral blood cells (white blood corpuscles) when the value of the maximum point is equal to or smaller than the reference value A while the cells S are identified as the cells (cancer cells) other than the peripheral blood cells when the value of the maximum value is greater than the reference value A. In this case, the cells S are identified by using only the value of the extreme point in the change of the transmitted light signal SG1. However, the value of the extreme point and the number of extreme points may be combined and the cells S may be identified by the value and the number.

Finally, on the basis of the identification result identified by the identifying unit 50, the cells S corresponding to the peripheral blood cells are sorted in a predetermined vessel (not shown in the drawings) when the peripheral blood cells are sorted, and the cells S corresponding to the cells other than the peripheral blood cells are sorted in a predetermined vessel when the cells other than the peripheral blood cells are sorted (step 6: S106). Then, the cell identification processing sequence using the cell identifying apparatus 10 according to the embodiment of the present invention ends.

In step 4: S104 described above, only the change of the transmitted light signal SG1 caused by the cells S is measured. However, transmitted light information corresponding to the peak, the width, and the area of the change of the transmitted light signal SG1 may be further measured, from the change of the transmitted light signal SG1. At this time, in step 5: S105 described above, the transmitted light information measured in step 4: S104 may be additionally used as a parameter of the identifying condition to identify the cells S.

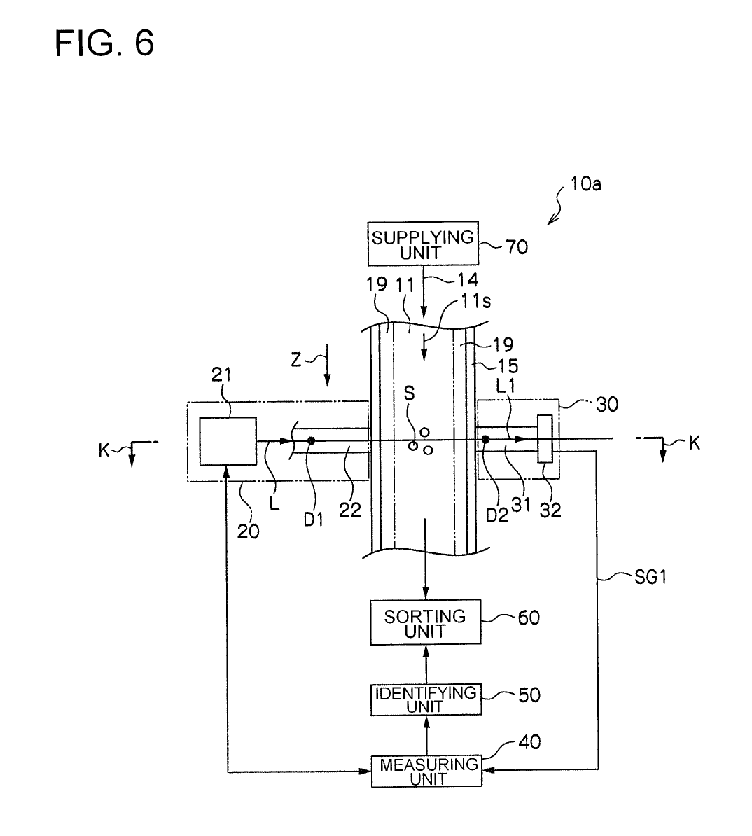

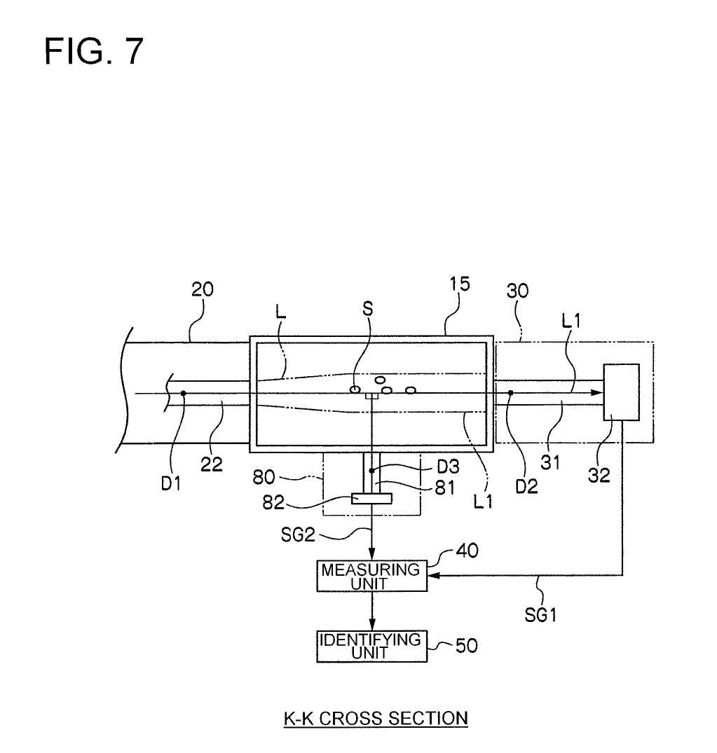

Next, another cell identifying apparatus according to an embodiment of the present invention will be described with reference to FIGS. 6 and 7.

FIG. 6 is a schematic side view of another cell identifying apparatus 10a according to the embodiment of the present invention. FIG. 7 is a cross-sectional view taken along the line K-K of FIG. 6.

Another cell identifying apparatus 10a according to the embodiment of the present invention shown in FIGS. 6 and 7 is different from the cell identifying apparatus 10 according to the embodiment of the present invention shown in FIGS. 1 and 2 in that another cell identifying apparatus includes a second light receiving unit 80 to receive lateral scattered light L2 of the cells S by the irradiation light L and output a scattered/fluorescent signal SG2 based on the lateral scatted light L2 to the measuring unit 40.

For example, as shown in FIGS. 6 and 7, the second light receiving unit 80 includes a light receiving optical fiber 81 and a light receiving element 82. As examples of the light receiving element 82, a photomultiplier tube and a photo detector are known. In this case, a third optical axis D3 of the second light receiving unit 80 has an angle of about 90 degrees with respect to the first optical axis D1 of the irradiating unit 20 and the second optical axis D2 of the light receiving unit 30, and is disposed at approximately the center of a wall surface where the second light receiving unit 80 is positioned, on the same plane as the first optical axis D1 of the irradiating unit 20 and the second optical axis D2 of the light receiving unit 30. In this case, the second light receiving unit 80 receives the lateral scattered light L2 and outputs the scattered/fluorescent signal SG2 based on the lateral scattered light to the measuring unit 40. However, the second light receiving unit 80 may receive fluorescent light of the cells S and output a fluorescent signal based on the fluorescent light to the measuring unit 40.

The measuring unit 40 of the cell identifying apparatus 10a measures the change of the transmitted light signal SG1 caused by the cells S, based on the transmitted light signal SG1 transmitted from the light receiving unit 30, and transmits the measurement result to the identifying unit 50. The measuring unit 40 measures the change of the scattered/fluorescent signal SG2 caused by the cells S, based on the scattered/fluorescent signal SG2 transmitted from the second light receiving unit 80, and transmits the measurement result to the identifying unit 50.

The identifying unit 50 of the cell identifying apparatus 10a identifies the cells S, on the basis of the value of the extreme point in the change of the transmitted light signal SG1 caused by the cells S and the change of the scattered/fluorescent signal SG2 caused by the cells S, which are transmitted from the measuring unit 40. That is, the identifying unit 50 identifies whether the cells S are the peripheral blood cells (that is, white blood corpuscles) or the cells other than the peripheral blood cells. In this case, the cells S are identified on the basis of the value of the extreme point in the change of the transmitted light signal SG1 and the change of the scattered/fluorescent signal SG2 caused by the cells S. However, the cells S may be identified on the basis of the value of the extreme point and the number of extreme points and the change of the scattered/fluorescent signal SG2 caused by the cells S.

The measuring unit 40 measures only the change of the transmitted light signal SG1 caused by the cells S and the scattered/fluorescent signal SG2. However, the measuring unit 40 may further measure transmitted light information corresponding to the peak, the width, and the area of the change of the transmitted light signal SG1, based on the change of the transmitted light signal SG1, and measure transmitted light information corresponding to the peak, the width, and the area of the change of the scattered/fluorescent signal SG2, based on the change of the scattered/fluorescent signal SG2. For this instance, the identifying unit 50 may also use further information such as the transmitted light information and the scattered/fluorescent information measured by the measuring unit 40 as parameters of the identifying condition to identify the cells S.

As such, another cell identifying apparatus 10a according to the embodiment of the present invention can identify whether the cells S in the peripheral blood are the peripheral blood cells or the cells other than the peripheral blood cells without using the antibody/antigen reaction of the cell surface. Since the change of the scattered/fluorescent signal SG2 caused by the cells S is added, it can be more specifically identified whether the cells S are the peripheral blood cells or the cells other than the peripheral blood cells, as compared with the cell identifying apparatus 10 according to the embodiment of the present invention.

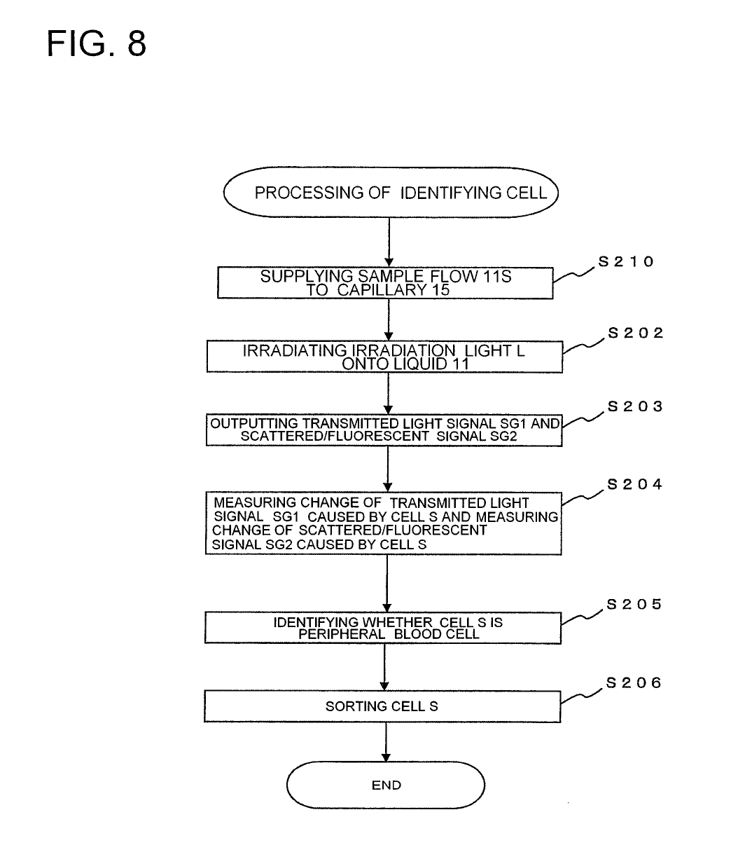

Next, a cell identification processing sequence using another cell identifying apparatus 10a according to the embodiment of the present invention will be briefly described with reference to FIG. 8.

FIG. 8 is a flowchart illustrating the cell identification processing sequence using another cell identifying apparatus 10a according to the embodiment of the present invention.

As shown in FIG. 8, first, the liquid 11 is supplied as the sample flow 11S from the supplying unit 70 to the capillary 15 in which the liquid 11 is obtained by adjustment such that the relative positions of the cells S in the liquid with respect to the irradiation light L from the irradiating unit 20 change at a constant speed (step 1: S201). The cells S are cells that are extracted from the peripheral blood where the preprocessing is performed.

Next, the irradiation light L from the irradiating unit 20 is irradiated onto the liquid 11 in the capillary 15 (step 2: S202). Next, the transmitted light L1 through the liquid 11 based on the irradiation light L is received by the light receiving unit 30 and the transmitted light signal SG1 based on the transmitted light is output to the measuring unit 40, and the lateral scattered light L2 of the cells S based on the irradiation light L is received by the second light receiving unit 80 and the lateral scattered light is output as the scattered/fluorescent signal SG2 to the measuring unit 40 (step 3: S203). In this case, the second light receiving unit 80 receives the lateral scattered light L2 and outputs the scattered/fluorescent signal SG2 based on the lateral scattered light to the measuring unit 40. However, the second light receiving unit 80 may receive fluorescent light of the cell S and output a fluorescent signal based on the fluorescent light to the measuring unit 40.

Next, the transmitted light signal SG1 output from the light receiving unit 30 is input to the measuring unit 40 and the change of the transmitted light signal SG1 caused by the cells S is measured by the measuring unit 40, and also the scattered/fluorescent signal SG2 output from the second light receiving unit 80 is input to the measuring unit 40 and the change of the scattered/fluorescent signal SG2 caused by the cells S is measured by the measuring unit 40 (step 4: S204).

Next, the cells S are identified on the basis of the value of the extreme point in the change of the transmitted light signal SG1 caused by the cells S and the change of the scattered/fluorescent signal SG2 caused by the cells S, which are measured by the measuring unit 40 (step 5: S205). That is, whether the cells S are the peripheral blood cells (that is, white blood corpuscles) or the cells other than the peripheral blood cells is identified by the identifying unit 50a. In this case, the cells S are identified on the basis of the value of the extreme point in the change of the transmitted light signal SG1 caused by the cell S and the change of the scattered/fluorescent signal SG2 caused by the cells S. However, the cells S may be identified on the basis of the value of the extreme point and the number of extreme points and the change of the scattered/fluorescent signal SG2 caused by the cells S.

Finally, on the basis of the identification result identified by the identifying unit 50, the cells S corresponding to the peripheral blood cells are sorted in a predetermined vessel (not shown in the drawings) when the peripheral blood cells are sorted, and the cells S corresponding to the cells other than the peripheral blood cells are sorted in a predetermined vessel when the cells other than the peripheral blood cells are sorted (step 6: S206). Then, the cell identification processing sequence using another cell identifying apparatus 10a according to the embodiment of the present invention ends.

In step 4: S204 described above, only the change of the transmitted light signal SG1 caused by the cells S and the scattered/fluorescent signal SG2 are measured. However, transmitted light information corresponding to the peak, the width, and the area of the change of the transmitted light signal SG1 may be further measured based on the change of the transmitted light signal SG1, and the scattered/fluorescent light information corresponding to the peak, the width, and the area of the change of the scattered/fluorescent signal SG2 may be further measured based on the change of the scattered/fluorescent signal SG2. At this time, in step 5: S205 described above, the transmitted light information and the scattered/fluorescent information measured in step 4: S204 may be additionally used as parameters of the identifying condition to identify the cells S.

### EXPLANATIONS OF LETTERS AND NUMERALS

10, 10a: cell identifying apparatus

11: liquid

11S: sample flow

15: capillary

19: sheath flow

20: irradiating unit

30: light receiving unit

40: measuring unit

50: identifying unit

60: sorting unit

70: supplying unit

80: second light receiving unit

S: cells

L: irradiation light

SG1: transmitted light signal

SG2: fluorescent/scattered signal

1. A cell identifying apparatus that identifies different cells or cell species in peripheral blood, comprising:
an irradiating unit that irradiates irradiation light onto a liquid in which cells in the peripheral blood are dispersed;
a light receiving unit that is provided at a position directly facing the irradiating unit, receives transmitted light through the liquid based on the irradiation light irradiated from the irradiating unit onto the liquid in a state in which relative positions of the cells with respect to the irradiation light change at a constant speed, and outputs a transmitted light signal based on the transmitted light;
a measuring unit that receives the transmitted light signal output from the light receiving unit and measures a change of the transmitted light signal, the change being caused by the cells; and
an identifying unit that identifies the cells in the peripheral blood, on the basis of a value of an extreme point in the change of the transmitted light signal, the change being caused by the cells and measured by the measuring unit.

2. The cell identifying apparatus of claim 1,
wherein, when a value of the transmitted light signal obtained based on the liquid having no cells is set as a reference value, the identifying unit compares a value of a maximum point in the change of the transmitted light signal caused by the cells and the reference value, and identifies the cells as cells other than peripheral blood cells when the value of the maximum point is greater than or equal to the reference value.

3. The cell identifying apparatus of claim 1 or 2,
wherein the cells that are identified as the cells other than the peripheral blood cells by the identifying unit are circulating cancer cells.

4. The cell identifying apparatus of any one of claims 1 to 3,
wherein the cells are cells on which fluorescent processing including an antibody/antigen reaction of a cell surface and expression of fluorescent proteins in the cells is performed.

5. The cell identifying apparatus of any one of claims 1 to 4,
wherein the measuring unit measures transmitted light information based on the change of the transmitted light signal, and
the identifying unit identifies the cells in the peripheral blood, on the basis of the transmitted light information measured by the measuring unit.

6. The cell identifying apparatus of any one of claims 1 to 5, further comprising:
a second light receiving unit that is provided at a position other than the position directly facing the irradiating unit, receives scattered light and/or fluorescent light of the cells based on the irradiation light irradiated by the irradiating unit, and outputs a scattered/fluorescent signal based on the scattered light and/or fluorescent light,
wherein the measuring unit receives the scattered/fluorescent signal output from the second light receiving unit and measures the change of the scattered/fluorescent signal caused by the cells, and
the identifying unit identifies the cells in the peripheral blood, on the basis of the change of the scattered/:fluorescent signal measured by the measuring unit.

7. The cell identifying apparatus of any one of claims 1 to 6,
wherein the measuring unit measures scattered/fluorescent information based on the change of the scattered/fluorescent signal, and
the identifying unit identifies the cells in the peripheral blood on the basis of the scattered/fluorescent information measured by the measuring unit.

8. The cell identifying apparatus of any one of claims 1 to 7,
wherein the irradiation light is non-condensed single-mode light.

9. The cell identifying apparatus of any one of claims 1 to 8, further comprising:
a sorting unit that sorts the cells in the peripheral blood identified by the identifying unit in a predetermined vessel.

10. A cell identifying method that identifies cells in peripheral blood using an irradiating unit configured to irradiate irradiation light onto a liquid in which the cells in the peripheral blood are dispersed, and a light receiving unit that is provided at a position directly facing the irradiating unit, receives transmitted light, and outputs a transmitted light signal based on the transmitted light to a measuring unit, the cell identifying method comprising:
(a) a step of irradiating irradiation light from the irradiating unit onto the liquid in a state in which relative positions of the cells with respect to the irradiation light change at a constant speed;
(b) a step of receiving transmitted light through the liquid based on the irradiation light by the light receiving unit and outputting a transmitted light signal based on the transmitted light to the measuring unit;
(c) a step of receiving the transmitted light signal output in the step (b) by the measuring unit and measuring the change of the transmitted light signal caused by the cells with the measuring unit; and
(d) a step of identifying the cells in the peripheral blood, on the basis of a value of an extreme point in the change of the transmitted light signal caused by the cells, which is measured in the step (c).

11. The cell identifying method of claim 10,
wherein, in the step (d), when a value of the transmitted light signal obtained based on the liquid having no cells is set as a reference value, a value of a maximum point in the change of the transmitted light signal caused by the cells and the reference value are compared, and the cells are identified as cells other than peripheral blood cells when the value of the maximum point is greater than or equal to the reference value.

12. The cell identifying method of claim 10 or 11,
wherein the cells that are identified as the cells other than the peripheral blood cells in the step (d) are circulating cancer cells.

13. The cell identifying method of any one of claims 10 to 12,
wherein the cells are cells on which fluorescent processing including an antibody/antigen reaction of a cell surface and expression of fluorescent proteins in the cells is performed.

14. The cell identifying method of any one of claims 10 to 13,
wherein, in the step (c), transmitted light information is measured based on the change of the transmitted light signal, and
in the step (d), the cells in the peripheral blood are identified on the basis of the transmitted light information measured in the step (c).

15. The cell identifying method of any one of claims 10 to 14,
wherein a second light receiving unit is further used, and the second light receiving unit is provided at a position other than the position directly facing the irradiating unit, receives scattered light and/or fluorescent light of the cells based on the irradiation light irradiated by the irradiating unit, and outputs a scattered/fluorescent signal based on the scattered light and/or fluorescent light to the measuring unit,
in the step (b), the scattered light and/or fluorescent light of the cells based on the irradiation light are/is received by the second light receiving unit and a scattered/fluorescent signal based on the scattered light and/or fluorescent light is output to the measuring unit,
in the step (c), the scattered/fluorescent signal output in the step (b) is received by the measuring unit and the change of the scattered/fluorescent signal caused by the cells is measured by the measuring unit, and
in the step (d), the cells in the peripheral blood are identified on the basis of the change of the scattered/fluorescent signal measured in the step (c).

16. The cell identifying method of any one of claims 10 to 15,
wherein, in the step (c), scattered/fluorescent information is measured based on the change of the scattered/fluorescent signal, and
in the step (d), the cells in the peripheral blood are identified on the basis of the scattered/fluorescent information measured in the step (c).

17. The cell identifying method of any one of claims 10 to 16,
wherein the irradiation light is non-condensed single-mode light.

18. The cell identifying method of any one of claims 10 to 17, further comprising:
(e) a step of sorting the cells in the peripheral blood identified in the step (d) in a predetermined vessel.
